(19) United States
(12) Patent Application Publication
(10) Pub. No.: US 2021/0092982 A1
(43) Pub. Date: Apr. 1, 2021

(54) MILLED FLAXSEED WITH TURMERIC AND PIPERINE

(71) Applicant: Virginia Health Food Limited, Carrigaline, Cork (IE)

(72) Inventors: Helen O'Dowd, Carrigaline (IE); Christin Schneider, Carrigaline (IE); Irene Gonda, Carrigaline (IE); Agnieszka Glebowska, Carrigaline (IE)

(21) Appl. No.: 16/772,236

(22) PCT Filed: Dec. 14, 2018
(86) PCT No.: PCT/EP2018/084955

§ 371 Date: Jun. 12, 2020

(30) Foreign Application Priority Data
Dec. 15, 2017 (GB) 1721045.1

(51) Int. Cl. A23L 3/3526; A23L 7/126; A23L 7/135; A61K 36/55; A23L 29/30; A23L 3/3472; A23L 33/00

(52) U.S. Cl. CPC A23L 3/3526; A23L 7/126; A23L 7/135; A23L 33/30; A23L 29/30; A23L 3/3472; A61K 36/55

(57) ABSTRACT

A mixture of milled flaxseed, turmeric powder and piperine for prolonging the shelf life of milled flaxseed.

### FIELD OF THE INVENTION

The invention relates to flaxseed and a method of extending its shelf-life. Specifically, the invention relates to a milled flaxseed and a method of extending its shelf-life.

### BACKGROUND TO THE INVENTION

Flax or Flaxseed or Flaxseed (Linum usitatissimum) is widely recognised in scientific and medical literature, and increasingly by health aware consumers, as a therapeutic food, due its (a) very significant amounts of Omega 3 (alpha-linoleic acid or ALA), which is an anti-inflammatory, (b) soluble fibre, which acts as a demulcent, soothing the digestive tract and (c) its high concentrations of lignans (phytoestrogens which act as hormone modulators in the body). These nutrients act synergistically and confer significant health benefits when used as part of a healthy diet and lifestyle. The health benefits include a reduction in cardiovascular diseases, decreased risk of cancer, particularly of the mammary and prostate glands, anti-inflammatory activity, laxative effect, alleviation of menopausal symptoms and prevention of osteoporosis. Flaxseed contains almost no digestible or glycemic carbohydrates. When consumed with carbohydrate-containing foods, its soluble fibre content delays gastric emptying, prolongs feelings of satiety and slows the release of sugars into the blood, making it an ideal addition to diabetes and weight management dietary protocols.

Because whole flaxseed has a tough outer coat that is not easily broken down in the body, consuming the whole seed has limited nutritional value. Milling the whole seed exposes the nutrients, including the Omega 3 (Alpha-Linoleic Acid, ALA), contained in the seed to the digestive process, so they are more bioavailable. However, exposing the contents of the seed by milling also risks promoting oxidation of the fatty acids in flaxseed and can limit the shelf life of the product. Suitable protective measures normally include removal of oxygen and flushing with inert gas (costly and with reduced line speeds), vacuum packaging (reduced line speeds and poor visual appearance of packaging), and addition of synthetic antioxidants such as Butylated hydroxyanisole (BHA) or Butylated hydroxytoluene (BHT) (not permitted under EU Regulation). However, gas flushing and vacuum packaging measures provide extended life only when pack is left un-opened. Once opened, the product must be used within a few days so that oxidative deterioration is kept to a minimum.

KR1020130078635 describes a mixture of flaxseed with turmeric, and also black pepper, among a plethora of varied combinations, for use as a health food supplement. US 2016008300 discloses a nutritional supplement composition comprising Co-Q10, fish oil, vitamin D and other additives such as flaxseed oil. US2003077336 describes a nutritional supplement comprising flaxseed oil, turmeric and other additives such as vitamins, minerals, sesame, ginger etc., for use as an anti-inflammatory complex. CA2737797 describes a formulation useful in a food supplement or health product comprising defatted flaxseed powder, turmeric and piperine. Other supplements available, such as TurmOil Supplements® capsules (from The Golden Paste Company), use flaxseed oil which lacks the fibre, protein and lignans contained in flaxseeds. Flaxseed oil has a high surface area and if it comes in contact with oxygen, it oxidises very quickly and thus turns rancid. Further, neither one of the prior art documents concern improving the product shelf life and efficacy

It is the object of the present invention to overcome at least one of the above-mentioned problems.

### SUMMARY OF THE INVENTION

The invention relates to the intimate combination of milled non-defatted flaxseed with fat soluble curcumin from natural whole turmeric root. The antioxidant properties of curcumin prolong the shelf life of the milled non-defatted flaxseed by delaying the primary oxidation of its lipids, thus enhancing the nutritional value of the whole product. This allows the inventors to bring a natural product to the market, (without the need to remove oxygen from the pack) that is affordable, with a shelf life sufficient to reach the consumer in top condition, with possible therapeutic benefits. Studies have indicated that turmeric oil, present in whole turmeric root, can enhance the bioavailability of curcumin. The synergies expected from this mixture include better bioavailability of ALA from the milled flaxseed in association with the curcumin. Further, curcumin in combination with ALA increases the conversion of the latter to EPA (eicosapentaenoic acid) and DHA (docosahexaenoic acid). Inclusion of piperine in the product is expected to enhance the effectiveness of the curcumin when ingested.

According to the present invention there is provided, as set out in the appended claims, a nutritional supplement composition comprising milled non-defatted flaxseed, turmeric and piperine.

There is also provided a composition for use in prolonging the shelf life of milled flaxseed, the composition comprising milled non-defatted flaxseed, turmeric and piperine.

In one embodiment, the composition or nutritional supplement composition comprises 30.00-99.499% milled non-defatted flaxseed, 0.5-10% turmeric and 0.001-0.01% piperine.

Preferably, the composition or nutritional supplement composition comprises 89.99-99.499% milled non-defatted flaxseed, 0.5-10% turmeric and 0.001-0.01% piperine.

Preferably, the composition or nutritional supplement composition further comprises flavouring, natural sweeteners (honey syrup, date syrup, raw cane syrup, brown rice syrup. agave syrup, yacon syrup, coconut sugar), natural sugar substitutes (inulin, stevia, xylitol, erythritol), seeds, plant protein powders, granules of freeze-dried yoghurt, kefir and a probiotic, nuts, nut flour, algal flour, plant oil preparations, green tea extract, herbal extract, olive leaf, moringa, baobab, monk fruit, vitamins, minerals, chlorella, spirulina, wheatgrass, barley grass, alfalfa, maca, Cacao, herbs and spices and their extracts, natural colouring, botanical extracts, plant stanols, stanol esters, beta-glucan, dried fruits, vegetables, a freeze-dried component, an air-dried component, aloe vera and its preparations, or a combination thereof.

Preferably, the milled non-defatted flaxseed is present as 94.995%.

Preferably, the turmeric is present as 5%. Preferably, the turmeric contains 6.5% curcumin.

Preferably, the piperine is present as 0.005%.

In an aspect, the flaxseed is organic or non-organic flaxseed. In an aspect, the turmeric is organic turmeric or inorganic turmeric. In one aspect, the piperine is organic piperine or inorganic piperine.

Preferably, the turmeric is present as dried turmeric root powder.

According to the appended claims, there is provided a method for prolonging the shelf life of milled non-defatted flaxseed, the method comprising adding 0.5-10% of turmeric and 0.001-0.01% of piperine in the finished blend of milled flaxseed turmeric and piperine.

There is also provided a method for prolonging the shelf life of a composition comprising milled non-defatted flaxseed, the method comprising adding to the composition 0.5-10% of turmeric and 0.001-0.01% of piperine in the finished composition comprising non-defatted milled flaxseed, turmeric and piperine.

Preferably, the milled flaxseed is present as 89.99-99.499%.

Preferably, the turmeric is present as 5%. Preferably, the turmeric contains 6.5% curcumin.

Preferably, the piperine is present as 0.005%.

Preferably, the flaxseed is organic flaxseed. Preferably, the turmeric is organic turmeric. Preferably, the piperine is organic piperine.

Preferably, the turmeric is present as dried turmeric root powder.

There is also provided a composition for use in improving the health of an individual by oral administration of the composition or nutritional supplement described above.

There is also provided a nutritional supplement comprising the composition described above. The nutritional supplement further comprises one or more of an air-dried, a freeze-dried component, a seed, a nut, a dried fruit, a vegetable, algal flour, a plant protein powder, a plant oil preparation, or a combination thereof.

Preferably, the air-dried and freeze-dried component is a fruit or a material derived from strawberry, apple, orange, apricot, grape, peach, pear, berries (raspberry, blueberry, blackberry), mango, melon, banana, citrus fruits, or a combination thereof. Preferably, the air-dried or freeze-dried component is a vegetable or product comprising one or more of asparagus, pea pods, peas, broccoli, cauliflower, mixed vegetables, yellow red or green peppers, lotus fruit, celery, garlic, onion, lettuce, carrot, potato, corn or a combination thereof.

Preferably, the seeds are selected from sunflower seeds, flaxseeds, pumpkin seeds, chia seeds, hemp seeds, sesame seed, melon seed, grapefruit seed, milk thistle seeds, sweet and bitter apricot seeds, quinoa seeds amaranth seeds, or a combination thereof. The seeds may be milled or whole. Preferably, the nuts are selected from cashew, almond, walnut, macadamia, peanut, hazelnut, pistachio, pecan, or a combination thereof. The nuts may be milled, chopped or whole. Preferably, the dried fruits are selected from strawberry, apple, orange, apricot, grape, peach, pear, berries (blueberry, blackberry), mango, melon, raspberry, banana, citrus fruits, or a combination thereof. Preferably, the vegetable is selected from asparagus, pea pods, peas, broccoli, cauliflower, mixed vegetables, yellow red or green peppers, lotus fruit, celery, garlic, onion, lettuce, carrot, potato, corn or a combination thereof.

Preferably, the algal flour is produced from chlorella, spirulina, marine algae (red, green or brown algae or seaweed), or combinations thereof.

Preferably, plant protein powders are selected from pea, rice, hemp, soya, sunflower seeds, pumpkinseeds, defatted nuts, or combinations thereof.

Preferably, plant-sourced oil preparations are oils derived from leaves, fruits, seeds or roots and rhizomes of plants selected from olive, neem, avocado, acai, rapeseed, orange, coconut, lemon, lime, hemp, melon, cranberry, cocoa, carob, blackcurrant, blackberry, evening primrose, borage, nigella sativa, apricot, apple seed, papaya, perilla, pomegranate, poppyseed, rice bran, thistle seed (Silybum Marianum), tigernut, sacha inchi, wheatgerm, or the like.

There is also provided a foodstuff comprising the composition described above. The foodstuff is selected from cereal bars and cereal snacks, granola bites, toppings for porridge, cereals and yogurts, flaxseed coatings for snack products, and nutritional supplements.

In an aspect, the non-defatted flaxseed is a microbially-reduced, non-defatted flaxseed.

### Definitions

In the specification, the terms “flaxseed”, “flax” and “flaxseed” should be understood to mean the seeds from Linum usitatissimum. Each term can be used interchangeably with the others. It should be understood that the flaxseed used in the invention is milled but remains non-defatted, that is, it still contains the whole amount of oil and Omega-3 as is present in the whole, unmilled seed. Flaxseed is also known as Linseed.

In the specification, the term “botanicals” should be understood to be extracts from plants such as β-carotene, astaxanthin, zeazanthin, lutein, lycopene.

In the specification, the term “phytochemicals” should be understood to mean chemical substances found in plants that have protective or disease-preventive properties when ingested by humans. Plants produce these chemicals naturally to protect themselves against bacterial, viral, fungal, and insect attack, or from the effects of too much sunlight. Some examples of phytochemicals are: lycopene, lutein, tannins, isoflavones, etc.

In the specification, the term “bioflavonoids” should be understood to mean a group of phytochemicals found in fruits, vegetables, nuts, seeds and many other plants that possess antioxidative, anti-inflammatory, antimutagenic and anticarcinogenic properties. As an example, tea flavonoids such as apigenin (from camomile) and epigallocatechin gallate (from green tea) can be used for the prevention of intestinal neoplasia, and cancer prevention in the gastrointestinal tract. Fruits, such as apples, onions and citrus fruits and berries, teas (camomile, green tea), vegetables (cabbage, broccoli), and herbs (parsley, celery) contain abundant amounts of bioflavonoids. They contain flavons (apigenin, luteolin), flavonols (quercetin, kaempferol), flavanols (epigallocatechin-3-gallate—EGCG), flavanons (naringenin, hesperidin) and isoflavons (genistein from soya), as well as anthocyanidins (plant pigment from berries, such as peonidin, malvidin, and petunidin, which are typically methylated).

In the specification, the term “plant stanol esters” should be understood to mean a heterogeneous group of phytosterol esters with a saturated sterol ring structure. Plant stanol esters are cholesterol-like molecules found in all plant foods (highest in vegetable oil) and are known to reduce the level of low-density lipoprotein (LDL) cholesterol in blood when ingested.

In the specification, the term “grass powders” should be understood to mean powdered wheatgrass, barley grass, oat grass, and the like. In cooler climates, the cereal grain (from wheat barley or oats) is planted in November and harvested in Spring just before the jointing stage. This ensures that it is at its peak nutritional content, as the plant is transitioning from vegetative to reproductive growth and accumulating substantial amounts of nutrients. Once cut, the grass is cleaned and chopped, dried under controlled conditions, and milled to a fine powder. The whole process is complete within 2 hours in order to preserve the nutritional value of the product.

In the specification, the term “algal powders” should be understood to mean the dried and milled powder of Macroalgae, i.e. seaweeds such as dulse and kelp, which are large multicellular algae; and Microalgae, i.e. unicellular algae such as chlorella, spirulina and blue green algae. Powder from microalgae contains 50 to 60 percent protein, but, unlike many isolated plant proteins such as soya, microalgae-algal powder is a whole-food ingredient and contains not only protein but other nutrients such as essential fats, DHA, fibre, vitamins A, B, C and E, and many minerals.

In the specification, the term “algal flours” should be understood to mean algae, such as chlorella, spirulina, marine algae, that are in the form of a flour. Algal flour is a lipid-rich, functional material harvested and dried from the above-mentioned algae grown under controlled conditions followed by fermentation. It is used to impart excellent mouthfeel and nutrition to a wide range of goods particularly baked goods, pasta, drink powders etc. and is nutritionally dense, being rich in essential fats, fibre, available protein and micronutrients. Many commercial companies produce algal flour, for example, Corbion Biotech Inc. (California), and Roquette (France).

In the specification, the term “flavouring substances” should be understood to mean chemical substances either naturally or synthetically derived which are added to food normally in combination with other flavouring substances in order to impart or modify its taste. Examples of such substances would be vanilla pastes, extracts and flavouring substances made with synthetic and natural vanillin; yeast and protein hydrolysates; products of the Maillard reaction between proteins or peptides and sugars etc. Other examples would be herb and spice oils and oleoresins and compound flavourings consisting of the following categories, flavouring substances, flavouring preparations, fruit and vegetable extracts, cheese powders and flavourings, meat flavourings, yeast extracts, spice and herb extracts, spices, herbs, oleoresins, thermal process flavourings, smoke flavourings, syrups, sweeteners, etc.

In the specification, the term “seed” should be understood to mean milled or whole flaxseed (including partially defatted flaxseed and microbially reduced flaxseed), chia seed, hemp seed, pumpkin seed, sunflower seed, sesame seed, melon seed, grapefruit seed, milk thistle seeds, sweet and bitter apricot seeds, quinoa seeds, amaranth seeds, or combinations thereof, including microbially reduced seeds.

In the specification, the term “nut” should be understood to mean peanut, almond, pistachio, walnut, macadamia, hazelnut, pecan, or combinations thereof. The nuts can be in the form of being whole, chopped or as a nut flour.

In the specification, the term “plant protein” should be understood to mean protein sourced from a plant or a part of a plant such as pea, hemp, rice, soya, sunflower seeds, pumpkinseeds, defatted nuts, or combinations thereof. The protein is generally in a powder form.

In the specification, the term “probiotic” should be understood to mean live micro-organisms which, when administered in adequate amounts, confer a health benefit on the host. Examples of such micro-organisms are, but not limited to, strains of lactic acid bacteria (LAB), such as those belong to the genera Lactobacillus (Lactobacillus casei, Lactobacillus salivarius, Lactobacillus brevis, Lactobacillus acidophilus). Bifidobacterium is another genera that has probiotic properties.

In the specification, the term “herbal extracts” should be understood to mean a material either liquid or solid which contains a portion of the original chemical contents of the plant, which may have therapeutic properties. Extracts can be made from roots, leavers flowers and bark, and the compounds found in these plant parts are extracted either by water, alcohol, hexane or other solvents, or by CO₂.

In the specification, the term “ambient” or “ambient temperature” should be understood to mean the temperature of the surrounding environment. For example, the ambient room temperature is generally 15° C. to 25° C.

In the specification, the term “vitamins” should be understood to mean nutraceutical additives in the form of Vitamins A, B, B1, B2, B6, B12, C, D, E, K, Niacin, Pantothenic acid, Folic acid, Biotin.

In the specification, the term “minerals” should be understood to mean nutraceutical additives in the form of minerals such as Calcium, Magnesium, Iron, Copper, Iodine, Zinc, Manganese, Potassium, Selenium, Chromium, Molybdenum, Phosphorus.

In the specification, the term “peroxide value” should be understood to mean a measure of the extent to which an oil sample has undergone primary oxidation, that is, the amount of peroxide oxide per kilogram of fat or oil. The unit of peroxide value is milliequivalents (mEq) or millimoles per kilogram.

In the specification, the term “organoleptically acceptable” should be understood to mean that the product is acceptable to the user via the senses (taste, smell and touch (texture)).

In the specification, the term “wt %” or “%” with reference to an amount of a component in the composition of ingredients should be understood to mean a weight percent of the total weight of the product. For example, 5% of product A in the composition corresponds to 5 g of product per 100 g of the composition.

In the specification, the term “foodstuffs” should be understood to mean cereal bars and cereal snacks, granola bites, toppings for porridge, cereals and yogurts, and nutritional supplements.

In the specification, the term “microbially reduced” or “microbial reduction” in reference to seeds should be understood to mean flaxseed which is pasteurised under conditions of time and temperature which ensure a 5-6 log reduction in microbial load, thus reducing the risk of food borne illness. Microbial load is measured by testing using standard laboratory microbial enumeration techniques covering typically Total Viable Count, Enterobacteria, Yeasts, Moulds, E. coli, Salmonella, Listeria, Staph. aureus. A typical microbial reduction process for flaxseed would be a saturated steam pasteurisation process carried out under vacuum which can be controlled to achieve the conditions necessary for the kill step while maintaining the chemical and sensory attributes of the flaxseed. Pasteurisation temperatures typically can range from 75° C. to 105° C. for typical time of 1 minute to 15 minutes depending on the product, the particle size of the product and how sensitive the components and composition of the components are to heat. For Flaxseed a 5 log reduction is achieved after just 1 minute at 75° C. The stored latent heat kills the bacteria on the seed surface when the steam condenses on it. The validation of the efficacy of the microbial reduction process is carried out when setting the process parameters This validation consists of setting up a microbiological challenge test. Samples of flaxseed that have seeded with a high concentration of bacteria e.g. E faecium are placed in several locations inside the batch. The batch is then processed and the samples are retrieved and analysed. A microbial reduction rate of >100,000 times (5 log reduction) is required for a successful pasteurisation. This procedure is repeated for three diferent pasteurisation runs to validate the Microbial reduction process.

The fat-soluble properties of curcumin are considered a significant advantage, as it enhances the ability of the turmeric to protect the highly unsaturated fats from oxidative damage when added to the milled flaxseed.

### BRIEF DESCRIPTION OF THE DRAWINGS

The invention will be more clearly understood from the following description of an embodiment thereof, given by way of example only, with reference to the accompanying drawings, in which:—

FIG. 1 illustrates the peroxide values (PV) of milled flaxseed and milled flaxseed mixed with turmeric stored at room temperature over a period of time. Peroxide Value (PV) testing determines the amount of peroxides in the lipids. Peroxides are the initial indicators of lipid oxidation which later continue to react producing secondary oxidation products which can result in unacceptable flavours and tainted product. FIG. 1 shows that Peroxides Values are significantly lower for flaxseed fortified with turmeric powder (5%). This means that adding turmeric powder to flaxseed results in lower amounts of peroxides in the flaxseed lipids both at the start of shelf life (timepoint 0) and at 9 months. Flaxseed on its own (100%) was more prone to initial oxidation which is manifested by higher PV than flaxseed combined with turmeric powder (1.6 vs. 0.09, 0.7 vs. 0.2). The amount of turmeric vs the amount of milled flaxseed used is: 5% turmeric powder, 94.995% milled flaxseed. 0.005% of piperine was also present in the milled flaxseed/turmeric mixture.

FIG. 2 illustrates the levels of free fatty acids (FFA) in milled flaxseed and milled flaxseed mixed with turmeric stored at room temperature over a period of time. Free Fatty Acids (FFA) testing determines the amount of fatty acids that have been liberated from their triglyceride (TG) structure. FFA are hydrolytic rancidity products and can produce strong flavours and odours at relatively low levels. FIG. 2 demonstrates higher FFA values for milled flaxseed on its own throughout the 9-month period. Addition of turmeric powder (5%) results in lower amounts of FFA being liberated from the TG structure of flaxseed lipids. In the present study, turmeric's preventive action against the FFA formation was particularly manifested at 9 months.

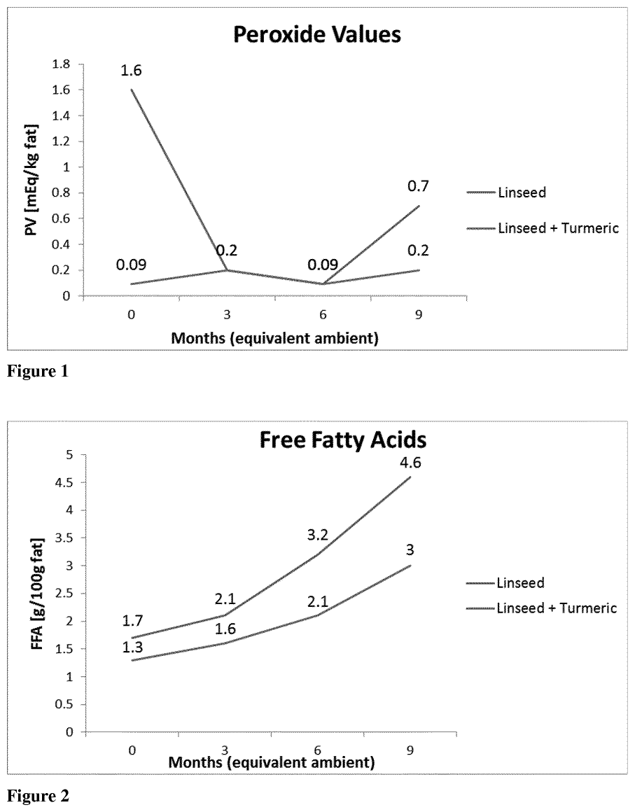

### DETAILED DESCRIPTION OF THE DRAWINGS

### Materials and Methods

The flaxseed is milled using a single pass method. In this method, the whole flaxseed is fed into a chamber incorporating a rotor and stator. The rotor consists of specially engineered flanges which propel the stream of flaxseed through a stator consisting of a grid of precisely engineered slots less than 1 mm in width with sharpened edges. The feed rate of the flaxseed and the speed of rotation of the flange determine the particle size distribution of the milled flaxseed. No other treatments are applied to the flaxseed following milling.

Organic turmeric powder (6.5% Curcuminoids) is obtained through curing, drying and polishing turmeric root. Curing usually takes place 2-3 days after harvesting and involves boiling fresh turmeric rhizomes in water until they are soft (approximately 45-50 min). The turmeric rhizomes, i.e. ‘fingers’, are then dried typically in the sun (which takes 10-15 days) or by using cross-flow hot air (max. 60° C., for 48 h). As dried turmeric has a poor appearance, it is then polished and smoothened. Finally, turmeric is milled to obtain 60-80 mesh powder. Organic turmeric powder can be obtained from, for example, Kräutermühle GmbH. The concentration of turmeric used in the compositions ranges from 0.5-10% of the composition (dry weight). The concentration used is controlled by concentration of curcuminoids required and with organoleptic acceptability issues if too much turmeric is added.

Piperine is produced from whole organic black pepper fruit. The black pepper fruit is quality inspected and cleaned, followed by crushing and extraction using organic ethanol. The liquid extract is then filtered and concentrated. The concentrate is further purified and precipitated and the precipitated material is centrifuged out. This is then vacuum dried, milled and sifted to produce piperine having 95% purity. Piperine is produced commercially by, for example, Vidya Europe. Piperine is typically used at a concentration of 0.001-0.01% of the composition (dry weight).

Other additives which may be used in the mix are marine calcium, sources of chelated minerals such as zinc and selenium, vitamin D (Source: Lanolin), beta-carotene (vegetable derived), plant stanol esters (Source: Vegetable oils), lecithins (Source: soya or sunflower oils), botanicals, phytochemicals and bioflavonoids (fruit and vegetable derived).

Dried fruits and vegetable pieces or extracts can be used also, along with grass powders, algal powders, seaweed extracts (such as fucoidan etc.), maca root, cherry extracts, cacao powders etc. Flavouring substances can also be used to provide more acceptable flavour profiles.

### Accelerated Shelf Life Trials

Storage at 37° C. under controlled conditions were conducted on:
1. Organic plain milled Flaxseed (Control).
2. Organic Milled Flaxseed with 5% organic turmeric root powder (6.5% curcuminoids) and 0.005% piperine.

### Measurement of Peroxide Value and Free Fatty Acids

Measurements were taken at the following time points:
Time Point 1—Start of Trial.
Time Points 2—at 3 weeks at accelerated conditions (Equivalent to 3 months storage at ambient conditions).
Time Point 3—at week 7 (equivalent to 6 months storage at ambient conditions).
Time Point 4—at week 10 (equivalent to 9 months storage at ambient conditions).
The samples were stored at 37° C. The rationale for the accelerated shelf life tests is that each 10° C. increase in storage temperature above ambient doubles the rate of chemical reactions, such as oxidation. Therefore, 37° C. provides a 4-fold increase in reaction rate. Ambient temperature is regarded as being room temperature varying between 15°−25° C. (59°−77° F.).
The samples were incubated at 37° C. and analysed for indicators of rancidity, namely PV and FFA, at Time Points 1 to 4. Additionally, odour assessment was conducted at each of the timepoints to evaluate any changes in odour and flavour profile against the control (stored frozen, then thawed and tested).

12 samples of each product were provided to the United Kingdom Accreditation Service (UKAS) accredited laboratory—3 samples (2 test samples and 1 frozen control) for each timepoint. PV and FFA were measured by UKAS accredited test method C-TM-028.

For Peroxide Value, hydroperoxides in the fat or lipid extract are reacted with potassium iodide and the liberated iodine titrated with sodium thiosulphate using an autotitrator.

For Free Fatty Acids (FFA), The acidic components in the fat are titrated with standard alkali and the result expressed as oleic acid.

All analyses are carried out using validated methods which meet strict performance criteria and are valid for the analysis of peroxide value and free fatty acids in raw materials and food products. Both tests are included in the UKAS accreditation schedule for the laboratory which conducted the analysis.

### Results

Please note that the units used for the ingredient composition are g/100 g or % of the composition. Please note that the units used for the product composition are g/100 g or % of the composition. Please note that the sum of all the parts of the composition make up 100 g or 100% of the composition.

Turmeric (Curcumin longa) has a long history of use as an herbal medicine and, being both an antioxidant and anti-inflammatory agent, is used for a wide variety of health conditions such as rheumatoid arthritis, conjunctivitis, skin cancer, wound healing, urinary tract infections, and liver disease. It is also used for digestive disorders, jaundice, menstrual difficulties, and colic; and for loss of appetite and liver and gallbladder complaints. In clinical practice, the main targets of turmeric action are the treatment of inflammatory bowel disease; and colon cancer. Curcumin (diferuloylmethane) is the yellow pigment present in turmeric and is considered its main bioactive component. It has been studied for its antioxidant, anti-inflammatory, anticancer, antiviral, and antibacterial activity in over 6,000 scientific citations.

TABLE 1
A typical nutritional food product described herein comprises:

| | Concentration Range % |
|---|---|
| Component | dry weight |
| Milled Flaxseed | 89.99 to 99.499 |
| Turmeric | 0.5 to 10 |
| (dried standardised root powder) | |
| Piperine | 0.001 to 0.01 |

The composition in Table 1 can be added as a component of a mixture containing one or more of the ingredients in Table 2.

TABLE 2
the typical nutritional food product can further comprise
one or more of the following ingredients:

| Component | Concentration Range % |
|---|---|
| Milled or Whole Chia seed | 0.1 to 10 |
| Hemp seed and other seeds | 0.1 to 60 |
| (pumpkin, sunflower, sesame, melon, etc.) | |
| Plant Protein Powders | 0.1 to 70 |
| Granules of freeze-dried yogurt, kefir and | 0.1 to 10 |
| probiotics. | |
| Almonds and other nuts (flour or chopped) | 0.1 to 50 |
| Air-dried Fruits and Vegetables | 0.1 to 60 |
| Freeze-dried fruits and vegetables | 0.1 to 40 |
| Plant oils | 0.1 to 5 |
| Algal flours (Chlorella, spirulina, | 0.1 to 20 |
| Marine algal extracts etc.) | |
| Herbs and spices | 0.1 to 10 |
| Flavourings and fruit oil preparations | 0.1 to 10 |
| Maca, cacao, moringa, baobab, | 0.1 to 20 |
| Green tea extracts, | |
| Herbal extracts, Olive leaf | |
| Vitamin and mineral preparations | 0.1 to 5 |
| Botanical extracts and functional food | 0.1 to 5 |
| colours i.e. β-carotene, | |
| astaxanthin, zeazanthin, lutein, lycopene. | |
| Beta Glucan, Plant stanols and stanol esters. | 0.1 to 10 |
| Natural sweeteners. | 0.001 to 30 |
| Fibres and fructo oligosaccharides (inulin) | 0.01 to 30 |
| Aloe vera and its preparations | 0.01 to 50 |

TABLE 3
The Product A (see Table 5 below) comprises:

| Component | Concentration % |
|---|---|
| Milled Flaxseed | 100 |

This is a control product.

TABLE 4
The Product B (see Table 5 below) comprises:

| Component | Concentration % |
|---|---|
| Milled Flaxseed | 94.995 |
| Turmeric (dried root powder) | 5 |
| Piperine | 0.005 |

The turmeric root used in Product B contains a curcumin concentration of 6.5%.

Analysis

TABLE 5

| | Equivalent | | | |
|---|---|---|---|---|
| | ambient | | Products | |
| Timepoint | (months) | Tests | Product A | Product B |
| 1 | 0 | PV [m Eq/ kg fat] | 1.6 | <0.1 |
| | | FFA [g/ 100g fat] | 1.7 | 1.3 |
| | | OA | bitter, nutty, | strong terpene/ |
| | | | no rancidity | herb, nutty |
| | | 03* [g/ 100g] | 16.8 | 16.7 |
| | | 06** [g/ 100g] | 5.01 | 4.9 |
| 2 | 3 | PV | 0.2 | 0.2 |
| | | FFA | 2.1 | 1.6 |
| | | OA\hat{ } | bitter, nutty, | bitter, strong |
| | | | no rancidity | herb/basil, no |
| | | | | rancidity |
| 3 | 6 | PV | <0.1 | <0.1 |
| | | FFA | 3.2 | 2.1 |
| | | OA | bitter, nutty, | Bitter, strong |
| | | | no rancidity | herb/basil, no |
| | | | detected | rancidity |
| | | | | detected |
| 4 | 9 | PV | 0.7 | 0.2 |
| | | FFA | 4.6 | 3 |
| | | OA | bitter, nutty, | Bitter, strong |
| | | | no rancidity | herb/basil, no |
| | | | detected | rancidity |
| | | | | detected |

*omega-3;
**omega-6;
\hat{ }organoleptic assessment

The results of the analysis illustrate that dried turmeric root (containing 6.5% curcumin), when mixed with milled flaxseed at 5% IFP (In Finished Product) has a significant delaying effect on the oxidative deterioration of the milled flaxseed. This delay in the deterioration of the quality and acceptability of the flaxseed is hugely significant. The intimate mixing of turmeric with milled flaxseed prior to packing, enables an increase in the shelf life attainable for milled flaxseed when conventionally packed for consumer use. The increase in shelf life attainable is due to the lower level of FFAs and oxidation end products produced in the product containing turmeric. It is acknowledged that the generation of higher levels of FFAs and oxidation end products are associated with flavour deterioration and poor organoleptic acceptability of high fat products.

Adding curcumin in the form of dried turmeric root at a level of about 5% of the dry weight of the root (giving a curcumin level of about 325 mg/100 g of the finished blend of milled flaxseed, turmeric and piperine) prevents or inhibits oxidation, even at elevated storage temperatures (37° C.) and in the presence of air, thus prolonging the shelf life of the milled flaxseed. Curcumin has been shown to have no toxicity, even in high dosages, and, once blended into the flaxseed, can be packed at the usual speeds and without vacuum packaging or inert gas flushing. The use of piperine and curcumin extends the shelf-life of milled flaxseed and increases the bioavailability of EPA and DHA.

In the specification, the terms “comprise, comprises, comprised and comprising” or any variation thereof and the terms “include, includes, included and including” or any variation thereof are considered to be totally interchangeable and they should all be afforded the widest possible interpretation and vice versa.

The invention is not limited to the embodiments hereinbefore described but may be varied in both construction and detail.

1. A composition for use in prolonging the shelf life of milled flaxseed, the composition comprising milled non-defatted flaxseed, turmeric and piperine.

2. A composition according to claim 1, wherein the composition comprises 30.00-99.499% milled non-defatted flaxseed, 0.5-10% turmeric and 0.001-0.01% piperine.

3. A composition according to claim 1, wherein the composition comprises 89.99-99.499% milled non-defatted flaxseed, 0.5-10% turmeric and 0.001-0.01% piperine.

4. A composition according to claim 1, further comprising one or more nutrients selected from flavouring, natural sweeteners (honey syrup, date syrup, raw cane syrup, brown rice syrup. agave syrup, yacon syrup, coconut sugar), natural sugar substitutes (inulin, stevia, xylitol, erythritol, glycosides), seeds, plant protein powders, granules of freeze-dried yoghurt, kefir, a probiotic, nuts, nut flour, algal flour, plant oil preparations, green tea extract, herbal extract, olive leaf, moringa, baobab, monk fruit, vitamins, minerals, chlorella, spirulina, wheatgrass, barley grass, alfalfa, maca, Cacao, herbs and spices and their extracts, natural colouring, botanical extracts, plant stanols, stanol esters, beta-glucan, a freeze-dried component, an air-dried component, aloe vera and its preparations, or a combination thereof.

5. A composition according to claim 1, in which the milled non-defatted flaxseed is present as 94.995%.

6. A composition according to claim 1, in which the turmeric is present at 5%.

7. A composition according to claim 1, in which when the turmeric is present as 5%, the turmeric contains 6.5% curcumin.

8. A composition according to claim 1, in which the piperine is present at 0.005%.

9. A composition according to claim 1, in which the flaxseed, turmeric, and piperine is organic or non-organic.

10. (canceled)

11. A composition according to claim 1, in which the turmeric is present as dried turmeric root powder.

12. (canceled)

13. A composition according to claim 1, in which the milled non-defatted flaxseed is microbially reduced.

14. A method for prolonging the shelf life of a composition comprising milled non-defatted flaxseed, the method comprising adding to the composition 0.5-10% of turmeric and 0.001-0.01% of piperine in the finished composition comprising non-defatted milled flaxseed, turmeric and piperine.

15. A method according to claim 14, in which the turmeric is present as 5%.

16. A method according to claim 14, in which when the turmeric is present at 5%, the turmeric contains 6.5% curcumin.

17. A method according to claim 14, in which the piperine is present at 0.005%.

18. A method according to claim 14, in which the milled non-defatted flaxseed, the turmeric and piperine is organic or non-organic flaxseed.

19. (canceled)

20. A method according to claim 14, in which the turmeric is present as dried turmeric root powder.

21. (canceled)

22. A method according to claim 14, in which the milled non-defatted flaxseed is microbially reduced.

23. A nutritional supplement or foodstuff comprising milled non-defatted flaxseed, turmeric and piperine.

24. (canceled)

25. The foodstuff of claim 23, wherein the foodstuff is selected from cereal bars and cereal snacks, granola bites, toppings for porridge, cereals and yogurts, and nutritional supplements.

26. (canceled)
